(19) United States
(12) Patent Application Publication
(10) Pub. No.: US 2022/0079684 A1
(43) Pub. Date: Mar. 17, 2022

(54) MEDICAL DEVICE FOR GUIDING A SURGICAL INSTRUMENT

(71) Applicants: Jaiben GEORGE, Chanagancherry (IN); George Pathiparampil JOLLY, Idukki (IN)

(72) Inventors: Jaiben GEORGE, Chanagancherry (IN); George Pathiparampil JOLLY, Idukki (IN)

(21) Appl. No.: 17/198,337

(22) Filed: Mar. 11, 2021

(30) Foreign Application Priority Data
Sep. 14, 2020 (IN) 202041039603

(51) Int. Cl. A61B 34/20

(52) U.S. Cl. CPC A61B 34/20; A61B 2034/2063

(57) ABSTRACT

The present invention relates to a medical device (100) for guiding and controlling movement of a surgical instrument (102). The medical device (100) includes a hollow tube (104) through which the surgical instrument (102) passes through to penetrate an anatomical structure, a plurality of ultrasonic probes arranged circumferentially at a distal end of the tube (104), the plurality of ultrasonic probes (108) adapted to move along a longitudinal axis of the surgical instrument (102) to accommodate one or more irregularities of a surface of the anatomical structure, a controller (110) adapted to process signals obtained from each of the plurality of ultrasound probes (108) and detect the one or more irregularities and depth of the surface of the anatomical structure, and a locking mechanism (112) to allow movement of the surgical instrument (102) toward the anatomical structure based on processing of the signal by the controller (110).

### TECHNICAL FIELD

The present invention relates to medical devices, and more specifically to a medical device for precisely guiding movement of a surgical instrument in orthopaedic/arthoscopic surgeries.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

In orthopedic surgery, for instance spine surgery, accurate placement of implants is essential for the success of any surgery. Majority of the spine involves insertion of one or more pedicle screws. These pedicle screws are inserted through pedicle of a vertebra which can be compared to block of bone. Currently, insertion of screws is done by making an entry point and a tract with use of a pedicle awl which is manually manipulated. The entry point is determined by the anatomy of the vertebral body and pedicle, and is largely dependent on the experience of the surgeon. Once the tract is made using an awl, the walls are inspected using a manual probe for any breach in the bony integrity of the walls. Once the walls are inspected and found to be intact, pedicle screw of appropriate size is inserted. As this method is user dependent, it is subject to errors, and breaches in the walls are common (as shown in FIG. 8A). Even if the pedicle awl is within the wall of the bone, it is possible for the tract to be eccentrically placed which can lead to break in the walls once the screw is inserted. A breach in the walls can lead to injury to vital structures like nerve roots, artery, or spinal cord. Moreover, loss of integrity in the walls can lead to decreased pull out strength of these screws which can lead to implant failures later on.

Further, medical procedures involving the vertebrae are normally complicated because of the preciseness and accuracy required to avoid both neural damage and injury to major blood vessels. Precision depth guided instruments are required to perform percutaneous spinal surgery. These surgeries sometimes require penetration of the hard cortical bone of the vertebra and traversal of the softer cancellous bone lying thereunder. A large force is normally required by a surgeon to penetrate the cortical bone. Once the cortical bone is penetrated, extreme care must then be taken to avoid rapidly penetrating through all of the cancellous bone.

There is also the danger of rapidly passing through the cancellous bone and then through the cortical bone on the other side of the vertebra. This can result in injury or damage to the spinal cord and/or other organs or blood vessels located adjacent the spine. In some instances, the force required to penetrate the cortical bone is greater than a surgeon can apply by hand. In these instances, a hammer or other similar instrument is required to force the instrument through the cortical bone. When a hammer or similar instrument is used, there is a greater danger of the instrument passing rapidly through the cancellous bone and out the other side of the vertebra.

Intra-operative fluoroscopy or computerized tomography (CT) has been used to ensure accurate position of screws. However, this results in use of heavy equipment which need to be repositioned constantly, and also leads to radiation exposure to patient and operating staff. Efforts have been made in the past focusing on devices based on ultrasound and electromagnetic signals to guide screw insertion in vertebral pedicles.

One such device using ultrasound to guide pedicle screw insertion uses ultrasound probes inserted into tip of a surgical instrument such as a drill or a guide wire so that its path can be continuously monitored by changes in ultrasound signals emitted from the probes. Although this is helpful in identification the location of the probe by telling the presence of the bone around the tip of the instrument, it can only be used as the tract is being made. Although such a device may provide real time information about the tract, it does not provide any information before the tract is made. Such a device has a disadvantage that multiple tracts might be made, before one with adequate bony wall around the tract is made. This leads to loss of unnecessary bone from the pedicle which might decrease pull-out strength of the screws.

Another such device which guides pedicle screw insertion includes a hand held device with ultrasound probes over a distal end. These probes help in identification of the trajectory of the drill or awl which is passed through the device.

However, such devices use ultrasound probes which are fixed at the distal end (either of the instrument or an outer tube thereof). The entry surface of the pedicle is often irregular, and requires a flexible arrangement of probes so that the track of the pedicle screw or awl is monitored in all directions. Moreover, such devices provide visual or auditory guidance about relative position of the instrument and the movement of the instrument is controlled manually by the user, which leads to inadvertent errors in positioning of the pedicle screw.

There is therefore a need to develop a medical device for accurately guiding a surgical instrument, which is capable of catering to the deficiencies associated with the conventional devices. The present invention has been made in view of the need for overcoming such deficiencies.

### SUMMARY OF THE INVENTION

The present invention relates to a medical device for accurately controlling movement of a surgical instrument. The medical device which uses information obtained from ultrasound signals to automatically control movement of the surgical instrument.

An aspect of the present invention pertains to a medical device for guiding a surgical instrument, including a hollow tube through which the surgical instrument passes through to penetrate an anatomical structure, a plurality of ultrasonic probes arranged circumferentially at a distal end of the tube, the plurality of ultrasonic probes adapted to move along a longitudinal axis of the surgical instrument to accommodate one or more irregularities of a surface of the anatomical structure, a controller adapted to process signals obtained from each of the plurality of ultrasound probes and detect the one or more irregularities and depth of the surface of the anatomical structure, and a locking mechanism to allow movement of the surgical instrument toward the anatomical structure based on processing of the signal by the controller.

According to an embodiment of the present invention, the plurality of ultrasonic probes are operatively coupled with a retention mechanism to allow movement of the plurality of ultrasonic probes along the longitudinal axis of the surgical instrument.

According to an embodiment of the present invention, the retention mechanism enables movement of the plurality of ultrasonic probes in directions perpendicular to the longitudinal axis of the surgical instrument.

According to an embodiment of the present invention, the tube is filled with an elastic material encircling the plurality of ultrasonic probes.

According to an embodiment of the present invention, each of the plurality of ultrasound probes includes an in-built transmitter for generating ultrasonic waves and a receiver for receiving signals reflected from adjacent surfaces of the anatomical structure.

According to an embodiment of the present invention, each of the plurality of ultrasound probes includes a transducer to convert the signals into electrical signals and transmit the electrical signals to the controller.

According to an embodiment of the present invention, the medical device further includes a battery to power the plurality of ultrasonic probes and the controller.

According to an embodiment of the present invention, the locking mechanism prevents movement of the surgical instrument toward the anatomical structure before the surgical instrument is properly aligned with the anatomical structure.

According to an embodiment of the present invention, the locking mechanism includes a plurality of projections arranged circumferentially on an inner portion of the tube to act as a lock for the surgical instrument passed through the inner portion of the tube.

According to an embodiment of the present invention, the controller processes the signals obtained from each of the plurality of ultrasound probes based on pre-determined threshold values.

According to an embodiment of the present invention, the tube includes a visual indicator unit at a proximal end. The visual indicator unit includes a plurality of circumferentially arranged visual indicators which provide information of the corresponding ultrasonic probes.

### OBJECTS OF THE PRESENT INVENTION

An object of the present invention is to provide a medical device capable of accurately guiding movement of a surgical instrument in orthopedic/arthoscopic operations.

Another object of the present invention is to provide a medical device employing a plurality of ultrasonic probes to precisely guide movement of the surgical instrument.

Another object of the present invention is to provide a medical device having auditory and/or visual indicators to facilitate accurate movement of the surgical instrument.

Still another object of the present invention is to provide a medical device for guiding accurate movement of the surgical instrument with minimal manual intervention.

Yet another object of the present invention is to provide a medical device which improves efficiency of an orthopedic/arthoscopic operation by using information obtained from ultrasound signals to automatically control movement of the surgical instrument.

### BRIEF DESCRIPTION OF ACCOMPANYING DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates an exemplary representation of a medical device for guiding and controlling movement of a surgical instrument in accordance with an embodiment of the present invention;

FIG. 2 illustrates an exemplary representation of the surgical instrument in accordance with an embodiment of the present invention;

FIG. 3 illustrates an exemplary representation of the surgical instrument passed through a hollow tube of the medical device in accordance with an embodiment of the present invention;

FIGS. 4A and 4B illustrate exemplary representations of arrangement of a plurality of ultrasonic probes at a distal end of the medical device in accordance with an embodiment of the present invention;

FIG. 5 illustrates an exemplary representation of the plurality of ultrasonic probes coupled with a retention mechanism in accordance with an embodiment of the present invention;

FIG. 6 illustrates an exemplary representation of a body of the medical device in accordance with an embodiment of the present invention;

FIG. 7 illustrates an exemplary representation of the surgical instrument in accordance with an embodiment of the present invention;

FIG. 8A illustrates the process of drilling followed by screw insertion into a pedicle; and

FIG. 8B illustrates an exemplary representation of vertebral bodies showing arrangement of one or more ultrasonic probes in accordance with an embodiment of the present invention.

### DETAILED DESCRIPTION OF THE INVENTION

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

If the specification states a component or feature “may”, “can”, “could”, or “might” be included or have a characteristic, that particular component or feature is not required to be included or have the characteristic.

Exemplary embodiments will now be described more fully hereinafter with reference to the accompanying drawings, in which exemplary embodiments are shown. This disclosure may however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Various terms as used herein are shown below. To the extent a term used in a claim is not defined below, it should be given the broadest definition persons in the pertinent art have given that term as reflected in printed publications and issued patents at the time of filing.

In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

The present invention relates to a medical device for accurately controlling movement of a surgical instrument. The medical device which uses information obtained from ultrasound signals to automatically control movement of the surgical instrument.

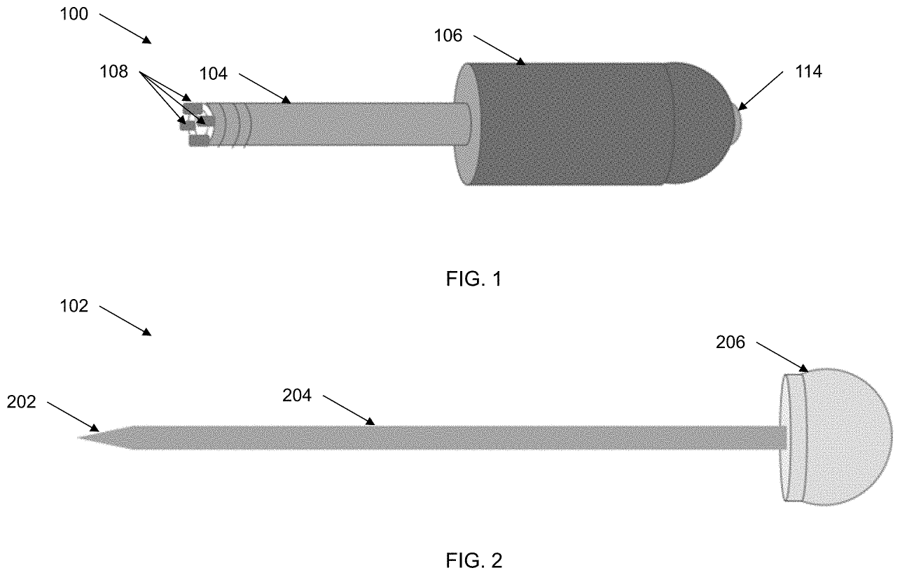

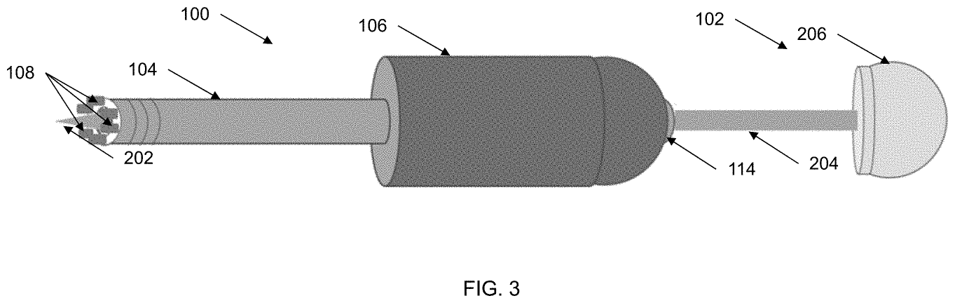

FIG. 1 illustrates an exemplary representation of a medical device (100) for guiding and controlling movement of a surgical instrument (102). FIG. 3 illustrates an exemplary representation of the surgical instrument (102) passed through a hollow tube (104) of the medical device (100). The medical device (100) may be employed in orthopedic/arthoscopic surgeries. The hollow tube (104) may have a cylindrical shape. In an embodiment, the hollow tube (104) may have a conical shape or any other shape as per requirement.

According to an embodiment of the present invention, the medical device (100) includes a hollow tube (104) through which the surgical instrument (102) passes through to penetrate an anatomical structure, as shown in FIG. 3. The hollow tube (104) is a flexible tube. The medical device (100) allows the surgical instrument (102) to accurately move in order to penetrate an anatomical structure, such as, spine, bone, cartilage, bone substitutes, allografts, etc. In an instance, the hollow tube (104) may have a length of, for example, 15 cm to 40 cm with the length of the surgical instrument (102) being 5 cm to 15 cm longer than the length of the tube (104). The hollow tube (104) may have a length larger than length of the surgical instrument (102). The hollow tube (104) extends to a body (106) of the medical device (100). The body (106) may be designed in a manner that its central portion is hollow in continuation with the tube (104) so that the surgical instrument (102) can pass through it.

According to an embodiment of the present invention, an inner diameter of the hollow tube (104) is about 3 mm to 10 mm with an outer diameter of the distal end and connecting segments being about 7 mm to 15 mm. The body (106) has an outer diameter of about 35 mm to 65 mm. In a preferred embodiment, the inner diameter of the hollow tube (104) is 4 mm with the outer diameter of the distal end and connecting segments being 10 mm, and the body (106) having an outer diameter of 50 mm.

According to an embodiment of the present invention, the medical device includes a plurality of ultrasonic probes (108) arranged circumferentially at a distal end of the hollow tube (104). The ultrasonic probes (108) are adapted to move along a longitudinal axis of the surgical instrument (102) to accommodate one or more irregularities of a surface of the anatomical structure.

According to an embodiment of the present invention, distal end of the hollow tube (104) is flexible and can adjust itself to contours of entry surface of the anatomical structure, such as, a pedicle insertion site. This ensures that all the ultrasound probes (108) are in contact with the bone or the anatomical structure, thereby improving accuracy of sonographic measurements. Each of the ultrasonic probes (108) is independent to each other, such that each ultrasonic probe (108) moves in the direction of the longitudinal axis of the surgical instrument (102) so that all the probes (108) are in contact with surfaces of the anatomical structure despite the irregularities thereof.

According to an embodiment of the present invention, the array of ultrasonic probes (108) are arranged in a circular fashion as the tube (104) is designed as a hollow cylinder. However, other shapes such as hexagon, square, etc. can also be used and in which case the arrangement of the ultrasound probes (108) differs accordingly.

According to an embodiment of the present invention, the ultrasound probes (108) are miniature probes having a quadrangular or circular cross-section with a diameter of about 2 mm to 3 mm, and a length of about 5 mm to 10 mm.

According to an embodiment of the present invention, the ultrasonic signals emitted from the probes travel through the anatomical structures directly in its path. These signals can travel through the outer cortical bone, underlying cancellous bones and get reflected from the second cortical surface. In some cases, the outer cortical bone at the insertion site might be removed in which case the signal travels through the cancellous bone and directly get reflected at the second cortical surface. The frequency used is dependent on the resolution of the images of the anatomical structure, and may be adjusted accordingly. As each probe (108) function independently and back scattering of signals emitted from one probe (108) might interfere with another, the medical (100) device has the option of different signal frequencies and or amplitudes for each probe (108), if required.

According to an embodiment of the present invention, the medical device further includes a controller (110) adapted to process signals obtained from each of the plurality of ultrasound probes (108) and detect the one or more irregularities and depth of the surface of the anatomical structure. The controller (110) is an electrical circuitry configured to process the signals obtained from each of the plurality of ultrasound probes (108) based on pre-determined threshold values.

According to an embodiment of the present invention, the medical device also includes a locking mechanism (112) to allow movement of the surgical instrument (102) toward the anatomical structure based on processing of the signal by the controller (110). The medical device (100) prevents manual intervention and further prevents slipping or change in trajectory of the surgical instrument (102) during the surgical operation, for instance, process of drilling of spinal bone. Further, the locking mechanism (112) of the medical device (100) prevents change in trajectory of the surgical instrument (102). The locking mechanism (112) may be programmed in such a way that only when the surgical instrument (102) is aligned with the surface of the anatomical structure, for instance, pedicle, based on the ultrasonic measurements, the surgical instrument (102) is allowed to advance. When at least one of the ultrasonic probes (108) suggest that the trajectory of the surgical instrument (102) is missing the pedicle, the locking mechanism (112) prevents any further advancement of the surgical instrument (102).

According to an embodiment of the present invention, the controller (110) may process the signals obtained from the probes (108) by analysing the signals in real-time. The signals may be processed by the controller (110) to detect the time taken for the signals to reach back as well as the amplitude of the returned signal which may be used to obtain depth of the surface of the anatomical structure, such as a bone, under the corresponding probe (108). The depths of bone underlying each probe (108) may be analysed by the controller (110) and the signals may be transmitted to various interfaces, such as, locking mechanism and visual indicator unit.

According to an embodiment of the present invention, for successful application of the medical device (100), appropriate indicators are required to guide how the position of the surgical instrument (102) needs to be changed. To serve this purpose, the medical device (100) has a visual or auditory indicator to notify the information on which direction of the surgical instrument (102) is to be changed to have an accurate position. The visual indicator unit (114) serve this purpose. The visual indicator unit (114) includes a plurality of circumferentially arranged visual indicators which provide information of the corresponding ultrasonic probes (108), , and each indicator denotes the signals received from the corresponding probe (108). This helps in identifying the probes/areas which are not in contact with a surface of the anatomical structure, such as a cortical bone. Therefore, the position of the surgical instrument (102) can be changed in the desired manner without any trial and error.

The visual indicator unit (114) is provided at the proximal end of the body (106) and includes multiple LED indicators arranged in circumferential fashion similar to the probes (108). The signals regarding the depth of the underlying bone from each probe (108) after being processed by the controller (110) may be used to visually indicate whether the position of that particular probe is satisfactory or not. When the surgical instrument (102) is ideally placed, there will be bone around all the probes, and all the indicators may be lit by an appropriate colour. When any of the probes (108) are not properly placed, that corresponding LED may be lit with a different colour. Thus, the visual indicator unit (114) guides the user to change the position or orientation of the surgical instrument (102) so that all the indicators are lit of the desired colour. There may also be colour coding if the readings are unreliable so that the user can make appropriately informed judgement.

Referring now to FIG. 2, where an exemplary representation of the surgical instrument (102) is shown. The surgical instrument (102) may be selected from the group including awl, drill, guide wire, K-wire, etc. which are used to surgical operations. The surgical instrument (102) may have a handle and an elongated tool which is passed through the hollow tube (104) to penetrate the surface of the anatomical structure, such as, bone, cartilage, bone substitutes, allografts, etc. The surgical instrument (102) may include a sharp tip (202) which may be conical, helical, pyramidal or any other shape depending on the structure intended to be penetrated. The surgical instrument (102) may have a solid shaft (204) which can be , or any other shape, of varying diameters to be passed through the tube (104), and has a handle of any suitable design (206).

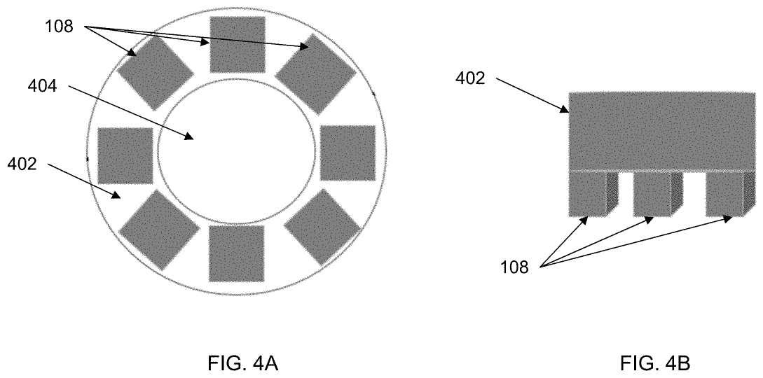

FIG. 4A shows an axial view and FIG. 4B shows a side view of distal end of the tube (102) showing arrangement of the ultrasound probes (108). In the depiction shown, the probes (108) may be arranged radially in a circular fashion. However, this configuration may be changed, and the number of probes (108) used can be flexible. The probes (108) may protrude distally from the tip as prongs, as shown in FIG. 4B. The material encircling the base (402) of the probes (108) may be made of material with elastic property so that mild translations in the orientation of the probes (108) is possible to accommodate the irregularities of the surface of the anatomical structure in contact. The opening (404) at the distal end of the tube (102) may be circular, as shown in FIG. 4A, but can have other shapes as well.

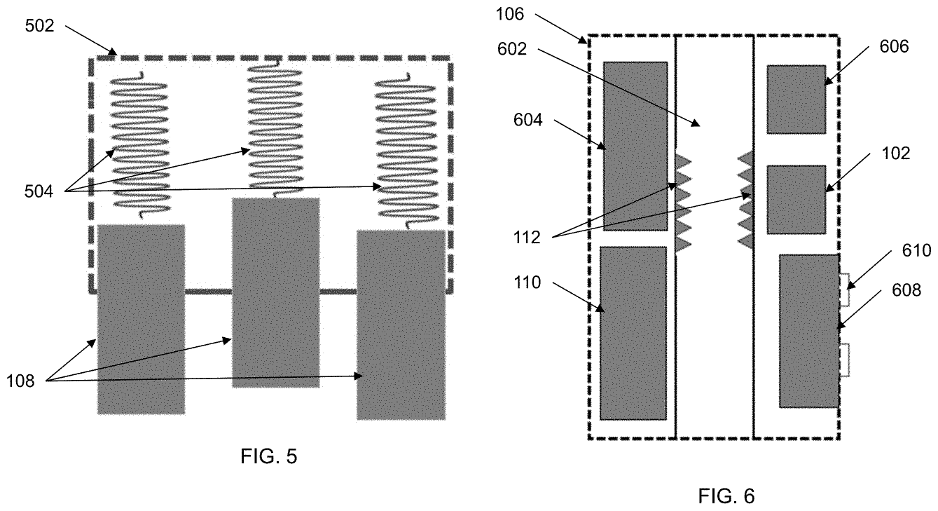

FIG. 5 illustrates an exemplary representation of the plurality of ultrasonic probes coupled with a retention mechanism in accordance with an embodiment of the present invention. FIG. 5 shows a representation of the distal end of the tube (104). The ultrasound probes (108) may be coupled to a base (502) with the help of springs (504) attached to their proximal ends. This retention mechanism allows significant movement of the probes (108) along the direction of the longitudinal axis of the surgical instrument (102), thus helping the probes (108) to be in contact with all the surfaces of the anatomical surface.

According to an embodiment of the present invention, the retention mechanism (502, 504) may include actuators, or any other mechanism, instead of springs to effect movement of the probes (108) along the direction of the longitudinal axis of the surgical instrument (102).

According to an embodiment of the present invention, the retention mechanism (502, 504) allows movement of the ultrasonic probes (108) along the longitudinal axis of the surgical instrument (102). According to another embodiment of the present invention, the retention mechanism (502, 504) enables movement of the ultrasonic probes (108) in directions perpendicular to the longitudinal axis of the surgical instrument (102). The tube (104) is filled with an elastic material encircling the ultrasonic probes (108) to account for mild translations in the orientation of the probes (108) to accommodate the irregularities of the surfaces of the anatomical structure in contact.

According to an embodiment of the present invention, each of the ultrasound probes (108) may include an in-built transmitter for generating ultrasonic waves, a receiver for receiving signals reflected from adjacent surfaces of the anatomical structure, and transducer to convert the signals into electrical signals and transmit the electrical signals to the controller (110).

According to an embodiment of the present invention, the controller (110) processes the signals obtained from each of the plurality of ultrasound probes (108) based on pre-determined threshold values, and detects the one or more irregularities and depth of the surface of the anatomical structure.

Referring now to FIG. 6, where an exemplary representation of the body (106) of the medical device (100) is shown. The body (106) may have a hollow central part (602) allowing a passage of the surgical instrument (102) to pass through. A portion of the hollow central part (602) may be designed in a way to accommodate the locking mechanism (112) which can act as an adjustable lock or pin to the surgical instrument (102) passed within the hollow central part (602) so as to prevent its advancement, if required. The body (106) may also contain at least a battery (604) to power at least the ultrasonic probes (108), the controller (110) and the interfaces of the medical device (100), the controller (110), a system for audio/visual processing (606), a system for transmitting mechanical signals to the locking mechanism (112), and a circuit (608) which may interact with the user through one or more controlling switches or a touch display (610).

According to an embodiment of the present invention, the locking mechanism (112) may include projections arranged circumferentially on an inner portion of the tube (104) to act as a lock for the surgical instrument (102) passed through the inner portion of the tube (104).

According to an embodiment of the present invention, the locking mechanism (112) acts as a safety feature to ensure that the surgical instrument (102) is only advanced when the medical device (100) is properly aligned with the anatomical structure. The locking mechanism (112) can be any sort of pin or lock which interlocks with the surgical instrument (102) so as to prevent its advancement. The locking mechanism (112) may be turned off using the controller (110) or the switches provided in the body (106). The body (106) may also contain additional switches to control light of the visual indicator unit (114), audio indicator, and the locking mechanism (112).

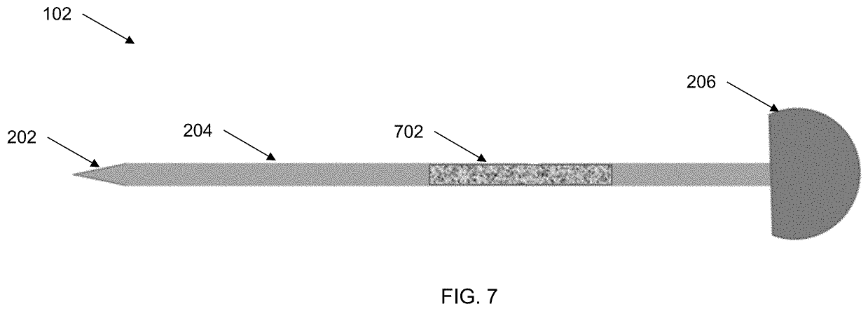

FIG. 7 illustrates an exemplary representation of the surgical instrument (102) in accordance with an embodiment of the present invention. In the present depiction, a simple hand held sharp tipped awl is shown. The surgical instrument (102) consists of a sharp tip (202) which may be conical, helical, pyramidal or any other shape depending on the structure intended to be penetrated. The surgical instrument (102) has a solid shaft (204) which can be , or any other shape, of varying diameters to be passed through the tube (104), and has a handle of any suitable design (206). A portion (702) of the shaft (204) may be modified in a way that it can interlock with the locking mechanism (112) of the medical device (100). Also, any routinely available drill, awl or guide wire (without specially designed locking mechanism) may be passed through the tube (104) and be used in the proposed design.

According to an embodiment of the present invention, the medical device (100) may be used with various surgical instruments (102), such as, a drill, an awl, guide wire, K-wire, an instrument for use in distal screw locking procedures, and the likes.

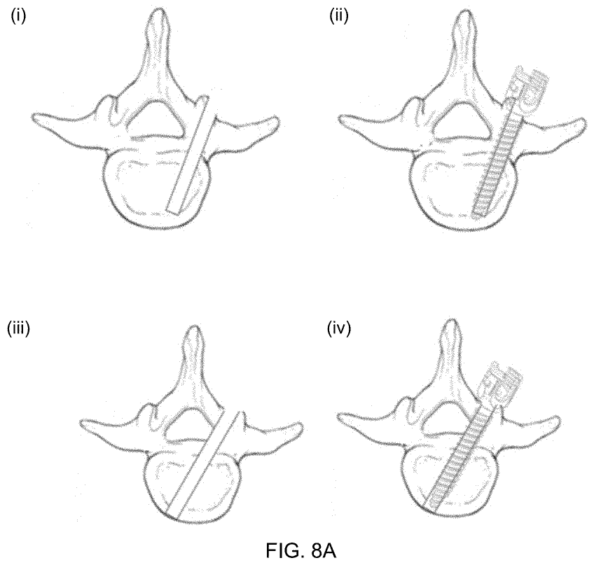

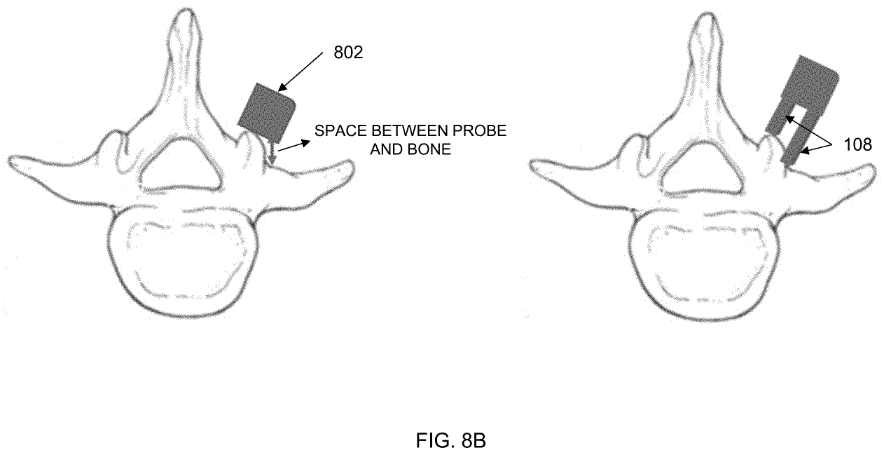

FIG. 8A illustrates a process of drilling a hole in a vertebral body followed by insertion of screw in the pedicle with a surgical instrument, such as, an awl or a drill. FIG. 8A(i) show a tract which is centrally located in the pedicle and which has adequate coverage of bone in all side, and FIG. 8A(ii) shows the screw inserted into the tract. FIG. 8A(iii) is an incorrectly placed tract where the medial pedicle wall is breached which can result in injury to important neural structures. FIG. 8A(iv) shows a screw placed in the incorrect tract.

FIG. 8B illustrates an exemplary representation of vertebral bodies showing arrangement of one or more ultrasonic probes of the medical device (100). On the left side, a normal probe/s (802) is shown which when placed on an irregular surface can lead to erroneous calculations as the entire probe (or set of probes) is not in contact with the surface of the anatomical structure. On the right side, the design shows multiple independent probes (108) whose lengths can be adjusted, all the probes (108) being in contact with the surface of the anatomical structure at any point of time.

The medical device (100) includes an outer hollow tube (104) which consists of tiny ultrasound probes (108) arranged circumferentially at the distal end/tip, which send signals to a controller (110) disposed in the body (106) of the device (100), where signals from the ultrasound probes (108) are processed based on pre-determined signal thresholds to send information signals to a user interface and mechanical interface (locking mechanism) regarding the position of the surgical instrument (102), for instance, a drill or an awl, passed within the hollow outer tube (104). The user interface consists of visual indicator unit (114) in a ring fashion which sends signals from the corresponding ultrasonic probes (108) so that the position and orientation of the surgical instrument (102) is automatically changed. The locking mechanism (112) is linked to the surgical instrument (102) in such a way that its movement will only occur after in pre-determined signals are received from the controller (110).

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

### ADVANTAGES OF THE PRESENT INVENTION

The present invention provides a medical device capable of accurately guiding movement of a surgical instrument in orthopedic/arthoscopic operations.

The present invention provides a medical device employing a plurality of ultrasonic probes to precisely guide movement of the surgical instrument.

The present invention provides a medical device having auditory and/or visual indicators to facilitate accurate movement of the surgical instrument.

The present invention provides a medical device for guiding accurate movement of the surgical instrument with minimal manual intervention.

The present invention provides a medical device which improves efficiency of an orthopedic/arthoscopic operation by using information obtained from ultrasound signals to automatically control movement of the surgical instrument.

We claim:

1. A medical device (100) for guiding a surgical instrument (102), comprising:
a hollow tube (104) through which the surgical instrument (102) passes through to penetrate an anatomical structure;
a plurality of ultrasonic probes arranged circumferentially at a distal end of the tube (104), the plurality of ultrasonic probes (108) adapted to move along a longitudinal axis of the surgical instrument (102) to accommodate one or more irregularities of a surface of the anatomical structure;
a controller (110) adapted to process signals obtained from each of the plurality of ultrasound probes (108) and detect the one or more irregularities and depth of the surface of the anatomical structure; and
a locking mechanism (112) to allow movement of the surgical instrument (102) toward the anatomical structure based on processing of the signal by the controller (110).

2. The medical device (100) as claimed in claim 1, wherein the plurality of ultrasonic probes (108) are operatively coupled with a retention mechanism (502, 504) to allow movement of the plurality of ultrasonic probes (108) along the longitudinal axis of the surgical instrument (102).

3. The medical device (100) as claimed in claim 2, wherein the retention mechanism (502, 504) enables movement of the plurality of ultrasonic probes (108) in directions perpendicular to the longitudinal axis of the surgical instrument (102).

4. The medical device (100) as claimed in claim 1, wherein the tube (104) is filled with an elastic material encircling the plurality of ultrasonic probes (108).

5. The medical device (100) as claimed in claim 1, wherein each of the plurality of ultrasound probes (108) comprises an in-built transmitter for generating ultrasonic waves and a receiver for receiving signals reflected from adjacent surfaces of the anatomical structure.

6. The medical device (100) as claimed in claim 5, wherein each of the plurality of ultrasound probes (108) comprises a transducer to convert the signals into electrical signals and transmit the electrical signals to the controller (110).

7. The medical device (100) as claimed in claim 1, wherein the locking mechanism (112) prevents movement of the surgical instrument (102) toward the anatomical structure before the surgical instrument (102) is properly aligned with the anatomical structure.

8. The medical device (100) as claimed in claim 1, wherein the locking mechanism (112) comprises a plurality of projections arranged circumferentially on an inner portion of the tube (104) to act as a lock for the surgical instrument (102) passed through the inner portion of the tube (104).

9. The medical device (100) as claimed in claim 1, wherein the tube (104) comprises a visual indicator unit (114) at a proximal end including a plurality of circumferentially arranged visual indicators which provide information of the corresponding ultrasonic probes (108).

10. The medical device (100) as claimed in claim 1, wherein the controller (110) processes the signals obtained from each of the plurality of ultrasound probes (108) based on pre-determined threshold values.
